(19) United States
(12) Patent Application Publication
(10) Pub. No.: US 2020/0164023 A1
(43) Pub. Date: May 28, 2020

(54) METHODS FOR PREVENTING AND TREATING INTRA-ABDOMINAL CANDIDIASIS

(71) Applicant: Cidara Therapeutics, Inc., San Diego, CA (US)

(72) Inventors: Kenneth BARTIZAL, La Jolla, CA (US); Paul DARUWALA, Del Mar, CA (US); Voon ONG, San Diego, CA (US); Taylor SANDISON, Encinitas, CA (US)

(21) Appl. No.: 16/636,083

(22) PCT Filed: Jan. 23, 2018
(86) PCT No.: PCT/US2018/014883

§ 371 Date: Feb. 3, 2020

Related U.S. Application Data
(60) Provisional application No. 62/540,704, filed on Aug. 3, 2017.

(51) Int. Cl. A61K 38/12; A61P 31/10

(52) U.S. Cl. CPC A61K 38/12; A61P 31/10

(57) ABSTRACT

Provided herein are methods of identifying an individual having or at risk of developing intra-abdominal candidiasis (1AC) and treating, mitigating, or preventing 1AC in the individual by administering a pharmaceutical composition including CD1 01, in salt or neutral form, in an amount sufficient to effectively treat or prevent 1AC.

### BACKGROUND

This invention features methods for the treatment or prevention of intra-abdominal candidiasis.

Intra-abdominal candidiasis (IAC) is a prominent invasive fungal infection associated with high mortality. Prompt source control and institution of antifungal therapy are major determinants of successful outcomes among patients with IAC. Echinocandin antifungal drugs are first-line agents for treating IAC, but their clinical effectiveness is highly variable with known potential for breakthrough resistance. Further, little is known about drug exposure at the site of infection. It has been postulated that restricted drug penetration into the abscesses or lesions characteristic of IAC is the main cause of antifungal treatment failure and creates a hidden reservoir of resistance to treatment. Thus, there is a need in the art for improved methods of preventing or treating IAC.

### SUMMARY OF THE INVENTION

The invention is directed to methods of preventing or treating intra-abdominal candidiasis (IAC) in an individual by administering CD101 in salt or neutral form.

In a first aspect, the invention features a method of treating an individual having intra-abdominal candidiasis (IAC). This method includes (i) identifying the individual as having IAC or suspected as having IAC; and (ii) administering to the individual a pharmaceutical composition comprising CD101 in salt or neutral form, wherein said composition is sufficient to treat the IAC.

In a second aspect, the invention feature a method of preventing IAC in an individual at risk thereof. This method includes (i) identifying the individual as being at risk of developing IAC; and (ii) administering to the individual a pharmaceutical composition comprising CD101 in salt or neutral form, wherein said composition is sufficient to prevent the development of IAC.

In any of the above embodiments, the subject may have an intra-abdominal abscess (IAA) and the pharmaceutical composition may be administered to the individual at a dosage and frequency sufficient for the concentration of CD101 within the IAA to exceed the mutant prevention concentration (MPC) of CD101 for Candida.

In some embodiments, the concentration of CD101 within the IAA achieves a concentration that exceeds the MPC within 6 hours following administration. In some embodiments, the concentration of CD101 within the IAA is maintained at a concentration that exceeds the MPC for at least 24 hours. In some embodiments, the concentration of CD101 within the IAA is maintained at a concentration that exceeds the MPC for at least 48 hours. In some embodiments, the concentration of CD101 within the IAA is maintained at a concentration that exceeds the MPC for at least 72 hours (e.g., at least 72 hours, at least 4 days, at least 5 days, at least 6 days, at least 7 days, at least 2 weeks, at least 3 weeks, at least 4 weeks, at least 5 weeks, at least 6 weeks, at least 8 weeks, at least 10 weeks, or at least 12 weeks). In some embodiments, the MPC is 1 μg/ml to 30 μg/ml (1.5 μg/ml±0.5 μg/ml, 2.5 μg/ml±0.5 μg/ml, 3 μg/ml±1 μg/ml, 4 μg/ml±1 μg/ml, 5 μg/ml±1 μg/ml, 6 μg/ml±2 μg/ml, 8 μg/ml±2 μg/ml, 10 μg/ml±5 μg/ml, 15 μg/ml±5 μg/ml, 20 μg/ml±5 μg/ml, or 25 μg/ml±5 μg/ml). In some embodiments, the MPC is about 16 μg/ml.

In any of the above embodiments, the pharmaceutical composition may be administered subcutaneously, intra-abdominally, or intravenously. In some embodiments, the pharmaceutical composition is administered weekly for one to twelve weeks (e.g., one, two, three, four, five, six, seven, eight, nine, ten, eleven, or twelve weeks) in an amount of about 50 mg to about 1200 mg (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg).

In any of the above embodiments, pharmaceutical composition may be administered is administered to the subject in a single dose, wherein the single dose comprises an amount of the CD101 in salt or neutral form sufficient to treat IAC. In some embodiments, the single dose includes from 50 mg to 1200 mg (e.g., 100±50 mg, 200±50 mg, 300±50 mg, 400±50 mg, 500±50 mg, 600±50 mg, 700±50 mg, 750±50 mg, 800±100 mg, 900±100 mg, 1000±100 mg, or 1100±100 mg) of the CD101 salt, or a neutral form thereof, administered orally, intravenously, subcutaneously, or intra-abdominally.

In any of the above methods, the pharmaceutical composition can consist of the CD101 salt, or a neutral form thereof, and the one or more pharmaceutically acceptable excipients.

In any of the above methods, the CD101 could be substituted with a compound described in U.S. Pat. No. 9,217,014, incorporated herein by reference. For example, CD101 could be substituted with a compound described in U.S. Pat. No. 9,217,014 selected from the group consisting of compound 6, compound 7, compound 12, compound 15, compound 17, compound 23, compound 24, and pharmaceutically acceptable salts thereof.

### Definitions

For the purpose of the present invention, the following abbreviations and terms are defined below.

As used herein, the term “about” refers to a range of values that is ±10% of specific value. For example, “about 150 mg” includes ±10% of 150 mg, or from 135 mg to 165 mg. Such a range performs the desired function or achieves the desired result. For example, “about” may refer to an amount that is within less than 10% of, within less than 5% of, within less than 1% of, within less than 0.1% of, and within less than 0.01% of the stated amount. Other than in the examples, or where otherwise indicated, all numbers expressing quantities of ingredients or reaction conditions used herein should be understood as modified in all instances by the term “about.”

As used herein, the term “CD101 salt” refers to a salt of the compound of Formula 1. CD101 has a structure (below) in which the tertiary ammonium ion positive charge of CD101 is balanced with a negative counterion (e.g., an acetate) in its salt form. The structure of CD101 is depicted below.

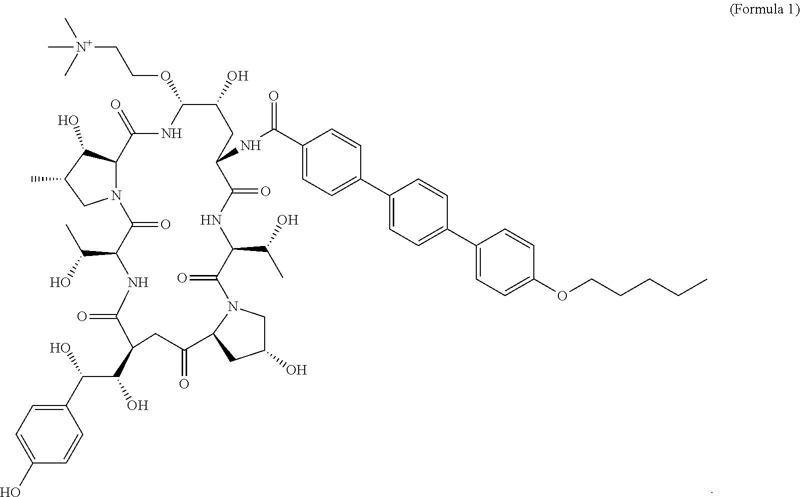 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)CC(=O)[C@@H]5C[C@@H](O)CN5C(=O)[C@H]([C@@H](C)O)NC4=O)cc3)cc2)cc1]

CD101 is a semi-synthetic echinocandin compound that inhibits the synthesis of 1,3-β-D-glucan, an essential component of the fungal cell wall of yeast forms of Candida species, Pneumocystis cysts, and regions of active cell growth of Aspergillus hyphae. The synthesis of 1,3-β-D-glucan is dependent upon the activity of 1,3-β-D-glucan synthase, an enzyme complex in which the catalytic subunit is encoded by FKS1, FKS2, and FKS3 genes. Inhibition of this enzyme results in rapid, concentration-dependent, fungicidal activity for, e.g., Candida spp.

The term “CD101 neutral form,” as used herein, includes the zwitterionic forms of CD101 in which the compound of Formula 1 has no net positive or negative charge. The zwitterion is present in a higher proportion in basic medium (e.g., pH 9) relative to CD101 or a salt of CD101. In some embodiments, the zwitterion may also be present in its salt form.

The “colonization” of a host organism includes the non-transitory residence of a fungi in or on any part of the body of an individual. As used herein, “reducing colonization” of a pathogenic fungi (opportunistic or non-opportunistic) in any microbial niche includes a reduction in the residence time of the pathogen and/or a reduction in the number (or concentration) of the pathogen in the colonized part of the individual's body.

By “concurrent antifungal treatment” is meant any additional dose of an antifungal agent (e.g., CD101 or another antifungal agent) administered within 12 hours, 24 hours, two days, three days, four days, five days, six days, or one week before or after administration of the single dose of CD101) that would confer therapeutic benefits (e.g., be systemically active) in the treatment of the targeted fungal infection (e.g., IAC) at the same time that CD101 is at a therapeutically effective concentration in the individual. In some instances, the single dose treatment is not combined with any other antifungal treatment within 1-21 days before or after administration. For example, a single dose of CD101 may be administered (e.g., intra-abdominally, orally, intravenously, subcutaneously, or intramuscularly) to an individual with a fungal infection (e.g., IAC) and the single dose effectively treats the fungal infection without necessitating additional antifungal treatments before, concurrently, or after the single dose treatment with CD101.

By “dose” is meant the amount of CD101 (Formula 1) administered to the individual.

The term “dosage form” or “unit dosage form,” as used herein, refers to physically discrete units suitable as unitary dosages, such as a pill, tablet, caplet, hard capsule or soft capsule, each unit containing a predetermined quantity of a drug.

By “effective” amount is meant the amount of drug required to treat or prevent a fungal infection (e.g., IAC) or a disease associated with a fungal infection (e.g., IAC). The effective amount of drug used to practice the methods described herein for therapeutic or prophylactic treatment of conditions caused by or contributed to by a fungal infection varies depending upon the manner of administration, the age, body weight, and general health of the individual. Ultimately, the attending physician will decide the appropriate amount and dosage regimen. Such amount is referred to as an “effective” amount.

By “infection” or “fungal infection” is meant a microbial dysbiosis (e.g., IAC) characterized by overgrowth or colonization of any part of the body of an individual by one or more species of fungi (e.g., fungal pathogens or opportunistic pathogens), reduction of which may provide benefit to the host. For example, the infection may include the excessive growth of or colonization by fungal species that are normally present in or on the body of an individual. Alternatively, the infection may include colonization by fungal species that are not normally present in or on the body of the individual. In some instances, the infection may include infection of a part of the body by a fungus that is indigenous to some parts of the human body (e.g., GI tract) but is detrimental when found in other parts (e.g., abdominal tissues beyond the GI tract). More generally, an infection can be any situation in which the presence of a microbial population(s) is damaging to a host body.

As used herein, “intra-abdominal candidiasis” or “IAC” refers to an intra-abdominal infection in an individual, wherein Candida is detected (e.g., detected using culture or non-culture approached) in a sample collected from an intra-abdominal site in the individual. IAC may be categorized under different classifications including, but not limited to, primary peritonitis, secondary peritonitis stemming from a GI tract source; intra-abdominal abscess (IAA) stemming from a GI tract source, secondary peritonitis stemming for a hepatobiliary or pancreatic source, IAA stemming from a hepatobiliary or pancreatic source, infected pancreatic necrosis, cholecystitis, or cholangitis.

As used herein, an “intra-abdominal abscess” or “IAA” refers to a localized pocket of infection that is walled-off from healthy tissue. In some instances, the IAA can result from (a) a pathologic process or breach of the GI tract or (b) a pathologic process of the liver, gallbladder, biliary or hepatic ducts, or pancreas. Infected bilomas, pancreatic pseudocysts, or other pancreatic or peripancreatic lesions are also categorized as abscesses. IAA may be identified, for example, by imaging studies (e.g., computed tomography scans) or by intra-operative examination.

As used herein, the term “minimum inhibitory concentration” or “MIC” refers to the concentration of an antifungal agent at which 50% to 100% growth inhibition of a fungus is achieved relative to a positive growth control. The method of establishing MIC will vary depending on the specific compound tested. For example, for echinocandins (e.g., CD101), MIC endpoints are determined after 24 hours incubation and correspond with the lowest drug concentration to produce a prominent decrease in turbidity (e.g., an approximately 50% reduction in growth relative to the drug-free growth control). For additional methods of determining the MIC of antifungal agents, see CLSI—Reference method for broth dilution antifungal susceptibility testing of yeasts; Approved-standard—Third Edition. CLSI document M27-A3. Wayne, Pa.: Clinical and Laboratory Standards Institute, 2008.

As used herein, the term “mutant prevention concentration” or “MPC” refers to the concentration of a drug sufficient to suppress the development of all but very rare spontaneous mutants. The range of drug concentrations between the MIC and MPC represents a mutant selection window wherein de novo mutants are most likely to occur. Therapeutic regimens that maximize the duration of drug concentrations in excess of the MPC thereby minimize the potential for resistance development during the course of therapy.

As used herein, “parenteral administration” and “administered parenterally” refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

As used herein, the term “salt” refers to any pharmaceutically acceptable salt, such as a non-toxic acid addition salt, metal salt, or metal complex, commonly used in the pharmaceutical industry. Acid addition salts include organic acids, such as acetic, lactic, palmoic, maleic, citric, cholic acid, capric acid, caprylic acid, lauric acid, glutaric, glucuronic, glyceric, glycocolic, glyoxylic, isocitric, isovaleric, lactic, malic, oxalo acetic, oxalosuccinic, propionic, pyruvic, ascorbic, succinic, benzoic, palmitic, suberic, salicylic, tartaric, methanesulfonic, toluenesulfonic, and trifluoroacetic acids, and inorganic acids, such as hydrochloric acid, hydrobromic acid, sulfuric acid, and phosphoric acid. Representative alkali or alkaline earth metal salts include sodium, lithium, potassium, calcium, and magnesium, among others.

As used herein, a “single dose” or “single dose treatment” of CD101 (e.g., CD101 in salt or neutral form) refers to treatment (e.g., substantial elimination) of a fungal infection (e.g., IAC) in an individual by administration of not more than one dose of a pharmaceutical composition including CD101 in salt or neutral form and one or more pharmaceutically acceptable carriers or excipients during a six week, 8 week, or 12 week period. Desirably, the single dose administration is sufficient to treat the fungal infection (e.g., IAC) without requiring a “concurrent antifungal treatment.”

By “individual” or “patient” is meant a human, non-human primate, or other mammal, such as but not limited to dog, cat, horse, cow, pig, turkey, goat, fish, monkey, chicken, rat, mouse, and sheep. An individual who is being treated for a fungal infection (e.g., IAC) is one who has been diagnosed by a medical practitioner as the case may be as having such a condition. Diagnosis may be performed by any suitable means. One in the art will understand that individuals of the invention may have been subjected to standard tests or may have been identified, without examination, as one at high risk due to the presence of one or more risk factors.

As used herein, the term “substantially eliminates” a fungal infection (e.g., IAC) refers to reducing colonization (see definition above) by one or more opportunistic or non-opportunistic pathogenic fungi (e.g., by about 1%, 2%, 5%, 10%, 20%, 30%, 40%, 50%, 60%, 70%, 80%, 90%, or %100 relative to a starting amount) in one or more parts of the body in an amount sufficient to restore a normal fungal population (e.g., approximately the amount found in a healthy individual) and/or allow benefit to the individual (e.g., reducing colonization in an amount sufficient to sustainably resolve symptoms). For example, a fungal infection can be caused by overgrowth of an opportunistic pathogen that is normally present on the individual's body but has grown above healthy levels, in which case the infection may be eliminated by reducing fungal species to a level typically found in a healthy individual without necessarily eliminating the fungal species. Alternatively, for example, a fungal pathogen or opportunistic pathogen may colonize a portion of the body in which it does not typically reside and thus, the infection is treated when the fungal population is eradicated.

As used herein, the term “substantially prevents’ refers to preventing increased colonization by one or more opportunistic or non-opportunistic pathogenic fungi (e.g., by about 1%, 2%, 5%, 10%, 20%, 30%, 40%, 50%, 60%, 70%, 80%, 90%, %100, or more than %100 relative to a starting amount) in one or more parts of the body in an amount sufficient to maintain a normal fungal population (e.g., approximately the amount found in a healthy individual), prevent the onset of a fungal infection (e.g., IAC), and/or prevent symptoms or conditions associated with infection. For example, individuals may receive prophylaxis treatment to substantially prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

As used herein, the term “treating” refers to administering a pharmaceutical composition for prophylactic and/or therapeutic purposes. To “prevent disease” refers to prophylactic treatment of an individual who is not yet ill, but who is susceptible to, or otherwise at risk of, a particular disease. To “treat disease” or use for “therapeutic treatment” refers to administering treatment to an individual already suffering from a disease to improve or stabilize the individual's condition. Thus, in the claims and embodiments, treating is the administration to an individual either for therapeutic or prophylactic purposes.

Other features and advantages of the invention will be apparent from the following detailed description, the drawings, and the claims.

### BRIEF DESCRIPTION OF THE DRAWINGS

The application file contains at least one drawing executed in color. Copies of this patent or patent application with color drawings will be provided by the Office upon request and payment of the necessary fee.

FIG. 1A shows the drug distribution of micafungin in infected liver tissues isolated from a mouse model of intra-abdominal candidiasis (IAC) after a single dose of micafungin. In the upper row are ion maps of micafungin in representative liver tissues collected at 1 hour, 3, 6, 24, and 48 hours following a single dose of micafungin at 5 mg/kg. The signal intensity color bar is fixed for micafungin, with gradually increasing intensity from blue (no signal) to red (max signal). Hematoxylin and eosin (H&E) staining and Gomori methenamine silver (GMS) staining of adjacent sections are shown below each set of ion maps. Outlines highlight the lesion area on each tissue section. Scale bars, 3 mm.

FIG. 1B shows the drug distribution of CD101 in infected liver tissues isolated from a mouse model of IAC after a single dose of CD101. In the upper row are ion maps of CD101 in representative liver tissues collected at 1 hour, 3, 6, 24, and 48 hours following a single dose of CD101 at 20 mg/kg. The signal intensity color bar is fixed for CD101, with gradually increasing intensity from blue (no signal) to red (max signal). Matched H&E and GMS staining results are shown in the middle and bottom rows, respectively. Scale bars, 3 mm.

FIG. 2 shows an enlarged view of drug penetration for micafungin at 24 hours, and CD101 at 6 and 48 hours following a single dosing in a mouse model of IAC. An enlarged view of drug distribution in a single lesion is shown at pixel level (upper level). Matched GMS staining of adjacent sections are shown in the bottom row. The signal intensity is fixed for CD101 and micafungin, respectively. Scale bars, 5 mm.

FIG. 3 shows the quantification of drug exposure in liver lesions and surrounding tissues isolated from a mouse model of IAC. Drug concentration was measured in lesions and surrounding uninvolved tissues dissected from liver sections collected at 6 and 24 hours following a single dose of micafungin at 5 mg/kg or CD101 at 20 and 5 mg/kg. Error bars, mean±standard deviation (s.d.) of three to five liver pieces or distinct lesions.

FIG. 4A shows drug penetration after multi-dosing micafungin. Micafungin steadily accumulates in abscesses upon two and three doses. Micafungin signal was only detected from lesion centers at steady state after three doses (upper row). H&E and GMS staining of adjacent sections are shown below each set of ion maps. Outlines highlight the lesion area on each tissue section. Scale bars, 3 mm.

FIG. 4B shows drug penetration after single dosing CD101. CD101 diffused into lesions thoroughly at 48 h post single dosing, and accumulated in necrotic area of each lesion at 72 hours. H&E and GMS staining of adjacent sections are shown below each set of ion maps. Outlines highlight the lesion area on each tissue section. Scale bars, 3 mm.

FIG. 5 shows a graph comparing drug accumulation in tissue isolated from a mouse model of IAC between multiple doses of micafungin (5 mg/kg) and a single dose of CD101 (20 mg/kg). Absolute drug level was measured for lesions and surrounding uninvolved tissues from liver samples collected at 48 hours and 72 hours following the first dose of micafungin, and those treated with a single dose of CD101 and collected at the matched time points. Error bars, mean±s.d. of three to five liver pieces or distinct lesions.

FIG. 6 shows the drug distribution of CD101 in infected kidney tissues isolated from a mouse model of IAC after a single dose of CD101. In the upper row are ion maps of CD101 in representative kidney tissues collected at 3, 6, 12, and 48 hours following a single dose of CD101 at 20 mg/kg. The signal intensity color bar is fixed for CD101, with gradually increasing intensity from blue (no signal) to red (max signal).

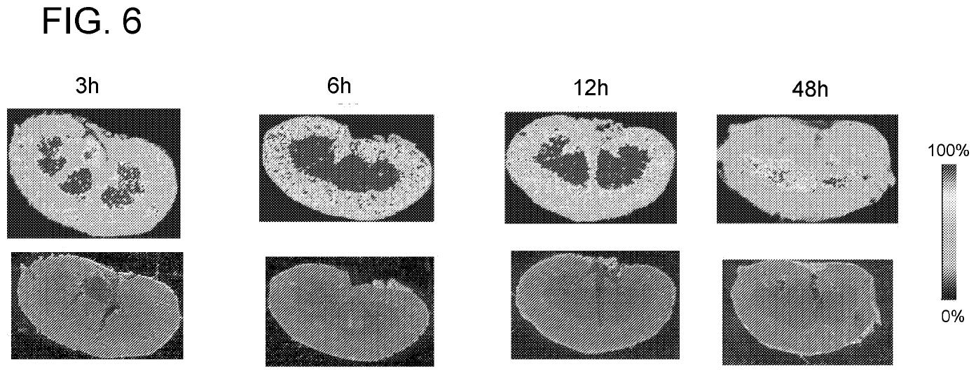

FIG. 7 shows the drug distribution of CD101 in infected kidney tissues isolated from a mouse model of IAC after a single dose CD101. In the upper row are ion maps of CD101 in representative kidney tissues collected at 3 and 12 hours following a single dose of CD101 at 10 mg/kg, 20 mg/kg, or 40 mg/kg. The signal intensity color bar is fixed for CD101, with gradually increasing intensity from blue (no signal) to red (max signal).

### DETAILED DESCRIPTION

Provided herein are methods of identifying an individual having or at risk of developing intra-abdominal candidiasis (IAC) and treating, mitigating, or preventing IAC in the individual by administering a pharmaceutical composition including CD101, in salt or neutral form, in an amount sufficient to effectively treat or prevent IAC.

I. Treatment Indications

Overview of IAC

The methods provided herein are indicated for use in the treatment or prevention of IAC in an individual in need thereof. IAC refers to an intra-abdominal infection, wherein a Candida spp. is detected (e.g., detected using culture or non-culture-based approaches) in a sample collected from an intra-abdominal site from the individual. IAC encompasses a range of disease manifestations that may occur in patients with various underlying conditions and risk factors involving the gastrointestinal (GI) tract and digestive system. The methods provided herein may be used to treat any classification of IAC. Different classifications of IAC include, but are not limited to:

(i) primary peritonitis (e.g., peritoneal inflammation associated with a recovery of Candida spp., occurring in the absence of an apparent breach of the GI tract or a pathologic process in a visceral organ);

(ii) secondary peritonitis stemming from a GI tract source (e.g., a peritoneal Candida infection resulting from a pathologic process or breach of the GI tract (stomach, small bowel, or colon), such as perforation, surgical leak, or trauma);

(iii) intra-abdominal abscess (IAA) stemming from a GI tract source (e.g., localized collection of Candida and pus that is walled-off from healthy tissue, resulting from a pathologic process or breach of the GI tract);

(iv) secondary peritonitis stemming for a hepatobiliary or pancreatic source (e.g., peritoneal Candida infection resulting from a pathologic process of the liver, gallbladder, biliary ducts, hepatic ducts, or pancreas);

(v) IAA stemming from a hepatobiliary or pancreatic source (e.g., an IAA resulting from a pathologic process of the liver, gallbladder, biliary ducts, hepatic ducts, or pancreas); (vi) infected pancreatic necrosis (e.g., Candida infection of non-vitalized pancreatic tissue resulting from chronic pancreatitis); or

(vii) cholecystitis or cholangitis (e.g., Candida infection of the gallbladder or biliary tract). IAC may also be classified as recurrent IAC (e.g., an intra-abdominal Candida infection occurring after an apparent resolution or clinical or radiographic findings of an initial Candida infection) or persistent IAC (e.g., infection continuing for ≥48 hours after appropriate source control and active antifungal treatment, for which Candida was re-isolated on culture form an intra-abdominal sample). See, e.g., Vergidis et al. (2016). PloS ONE. 11(4): e0153247, hereby incorporated by reference.

Further, the methods described herein may be used to treat or prevent IAC associated with infection by one or more Candida species, e.g., C. albicans, C. glabrata, C. dubliniensis, C. krusei, C. parapsilosis, C. tropicalis, C. orthopsilosis, C. guilliermondii, C. rugosa, C. auris, C. lusitaniae, C. utilis, or other Candida species. In some instances, the IAC may be associated with a Candida infection that is resistant to treatment with one or more antifungal drugs (e.g., azole compounds, echinocandins, polyene compounds, or flucytosine). In some instances, the IAC is associated with a Candida mono-infection. Alternatively, the IAC may be associated with a bacterial co-infection, wherein a Candida spp. and one or more bacterial species (e.g., Enterococcus, Enterobacteriaceae, or Klebsiella spp.) is detected in a sample collected from an intra-abdominal site from the individual.

Methods of Identification and Treatment

In one aspect, provided herein are methods for identifying and treating individuals having IAC, wherein the treatment comprises administration of a pharmaceutical composition including CD101 in salt or neutral form. In some instances, an individual may be identified as having IAC based on clinical evidence of an intra-abdominal infection and detection of Candida in a sample collected from the individual. The sample obtained from the individual may include, for example, a blood sample or an intra-abdominal specimen (e.g., purulent or necrotic intra-abdominal specimens) obtained during surgery or aspiration from the site of infection. Upon obtaining a sample from the individual, one or more methodologies known in the art may be used to assess the sample for evidence of a Candida infection. Diagnostic criteria to identify individuals having IAC can include culture and/or non-culture-based methods. Non-culture diagnostic methods include, e.g., antigen or antibody (e.g., mannan or anti-mannan IgG) detection, β-D-glucan detection assays, or polymerase chain reaction (PCR) assays to detect the presence of Candida. Culture based methods include, e.g., blood cultures or cultures of tissues or fluids recovered from infected sites. See, e.g., Bassetti et al. (2013). Intensive Care Med. 39:2092-2106. and Pappas et al. (2016) CID. 62(4):e1-50., hereby incorporated by reference. Abscesses and lesions are the predominant histopathological findings within abdominal organs from individuals with IAC. As such, the individual may be further assessed for the presence of lesions or IAAs using an imaging-based approach (e.g., computed tomography scan) or by intraoperative examination

In another aspect, provided herein are methods for identifying and treating individuals at risk of developing IAC, wherein the treatment comprises administration of a pharmaceutical composition including CD101 in salt or neutral form. Individuals at risk of developing IAC may be identified based on one or more risk factors. Examples of specific risk factors for IAC include, but are not limited to, abdominal surgery (e.g., surgeries involving the digestive or biliary tract within the preceding 12 months); gastrointestinal (GI) perforations (e.g., recurrent perforations and/or perforations untreated within 24 hours); gastrointestinal anastomosis leakage (e.g., upper GI tract leakage or lower GI tract leakage); or multifocal colonization by Candida spp. Individuals who may benefit from the treatments described herein may also be identified based on nonspecific risk factors including acute renal failure, central venous catheter placement, total parenteral nutrition, intensive care unit stay, sepsis, diabetes, immunosuppression, or prolonged broad-spectrum antibacterial therapy. Further risk factors for IAC include infection or inflammation resulting from conditions such as appendicitis, diverticulitis, Crohn disease, pancreatitis, pelvic inflammatory disease, traumatic abdominal injuries (e.g., lacerations and hematomas of the liver, pancreas, spleen, and intestines) or any condition causing generalized peritonitis.

Treatment of IAC using the methods described herein may further include one or more additional treatment methods. For example, an individual who is administered a pharmaceutical composition including CD101 in salt or neutral form according to the methods of the inventions may additionally receive appropriate source control interventions for IAC, including drainage of infected material, debridement of damaged tissue, and/or surgical correction of the underlying pathology (e.g., perforation or leak). In some instances, the source control is surgical intervention, percutaneous drainage, or transgastric drainage. In some instances, the treatment methods described herein may further involve administration of one or more additional anti-microbial agents (e.g., an antibacterial agent and/or antifungal agent) to the individual, as further described in section IV below.

II. Pharmaceutical Formulations

The pharmaceutical composition (e.g., including CD101 in salt or neutral form) of the methods described herein may be formulated for, e.g., intra-abdominal administration, intraoral administration, intravenous administration, intramuscular administration, intradermal administration, intraarterial administration, subcutaneous administration, oral administration, or administration by inhalation. In some instances, the pharmaceutical composition including CD101 (e.g., CD101 in salt or neutral form) may be formulated for intravenous administration. In some instances, the pharmaceutical composition including CD101 (e.g., CD101 in salt or neutral form) may be formulated for intra-abdominal administration.

The pharmaceutical composition including CD101 (e.g., CD101 in salt or neutral form) may be formulated with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The pharmaceutical composition may be in a unit dose form as needed. The amount of active component (e.g., CD101 in salt or neutral form) included in the pharmaceutical composition of the invention are such that a suitable dose within the designated range is provided (e.g., a dose of 50 to 800 mg or 500 mg to 1200 mg of CD101 in salt or neutral form).

Acceptable carriers and excipients in the pharmaceutical composition of the present invention are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The compositions may be formulated according to conventional pharmaceutical practice. The concentration of the compound in the formulation will vary depending upon a number of factors, including the dosage of the drug to be administered, and the route of administration.

In some instances, the pharmaceutical composition including CD101 (e.g., CD101 in salt or neutral form) may be formulated in the form of liquid solutions or suspensions and administered by a parenteral route (e.g., subcutaneous, intra-abdominal, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), F-12 medium). For injectable formulations, various effective pharmaceutical carriers are known in the art. See, e.g., Remington: The Science and Practice of Pharmacy, 22nd ed., (2012) and ASHP Handbook on Injectable Drugs, 18th ed., (2014).

In some instances, the pharmaceutical composition including CD101 (e.g., CD101 in salt or neutral form) can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium).

The pharmaceutical compositions of the invention can alternatively be formulated with excipients that improve the oral bioavailability of the compound. For example, the dosage forms of the invention can be formulated for oral administration with medium chain (C8 to C12) fatty acids (or a pharmaceutically acceptable salt thereof), such as capric acid, caprylic acid, lauric acid, or a pharmaceutically acceptable salt thereof, or a mixture thereof. The formulation can optionally include a medium chain (C8 to C12) alkyl alcohol, among other excipients. Alternatively, the compounds of the invention can be formulated for oral administration with one or more medium chain alkyl saccharides (e.g., alkyl (C8 to C14) beta-D-maltosides, alkyl (C8 to C14) beta-D-Gulcosides, octyl beta-D-maltoside, octyl beta-D-maltopyranoside, decyl beta-D-maltoside, tetradecyl beta-D-maltoside, octyl beta-D-glucoside, octyl beta-D-glucopyranoside, decyl beta-D-glucoside, dodecyl beta-D-glucoside, tetradecyl beta-D-glucoside) and/or medium chain sugar esters (e.g., sucrose monocaprate, sucrose monocaprylate, sucrose monolaurate and sucrose monotetradecanoate).

The methods disclosed herein may also further include the administration of an immediate-release, extended release, or delayed-release formulation of CD101 in salt or neutral form.

III. Dosage and Administration

The pharmaceutical compositions (e.g., including CD101 in salt or neutral form) of the methods described herein may be administered to the individual at a dosage and frequency sufficient to treat IAC. Abscesses and lesions associated with an invasive Candida infection are the predominant histopathological findings within abdominal organs from individuals with IAC. In some instances, to effectively target the Candida infection, the pharmaceutical compositions described herein may be administered to the individual at a dosage and frequency sufficient for CD101 to penetrate an abscess or lesion and be retained at the site of infection (e.g., within an IAA or lesion) at a concentration and for a length of time sufficient to substantially reduce or eliminate a Candida infection (e.g., reduce an intra-abdominal population of Candida in the individual by about 1%, 2%, 5%, 10%, 20%, 30%, 40%, 50%, 60%, 70%, 80%, 90%, or 100% relative to a starting amount).

The amount of drug (e.g., CD101 in salt or neutral form) that can effectively penetrate the site of infection (e.g., within an IAA or lesion) is one important consideration for treating IAC. For example, the pharmaceutical compositions described herein may be administered to the individual at a dosage and frequency sufficient for the concentration of CD101 at the site of infection (e.g., within an IAA or lesion) to exceed the mutant prevention concentration (MPC) of Candida. In some instances, the pharmaceutical compositions described herein are administered to the individual at a dosage and frequency sufficient for the concentration of CD101 at the site of infection (e.g., within an IAA or lesion) to be equal to or greater than an MPC of about 1 μg/ml to about 30 μg/ml (e.g., exceed about 1±0.5 μg/ml, 1.5±0.5 μg/ml, 2±1 μg/ml, 3±1 μg/ml, 4±1 μg/ml, 5±1 μg/ml, 6±1 μg/ml, 7±1 μg/ml, 8±1 μg/ml, 9±1 μg/ml, 10±2 μg/ml, 12±2 μg/ml 14±2 μg/ml, 16±2 μg/ml, 18±2 μg/ml, 20±2 μg/ml, 22±2 μg/ml, 24±2 μg/ml, 26±2 μg/ml, 28±2 μg/ml, or 30±2 μg/ml). In some instances, the concentration of CD101 at the site of infection (e.g., within an IAA or lesion) is equal to or greater than an MPC of about 16 μg/ml. In some instances, the pharmaceutical compositions described herein are administered to the individual at a dosage and frequency sufficient for the concentration of CD101 at the site of infection to achieve a concentration equal to or greater than the MPC of Candida within about 1 hour to about 24 hours (e.g., within about 30 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours). In some instances, the dosage and frequency of administration is sufficient for the concentration of CD101 at the site of infection (e.g., within an IAA or lesion) to achieve a concentration equal to or greater than the MPC within about 6 hours.

Another important consideration in the treatment of IAC is the length of time the drug (e.g., CD101 in salt or neutral form) is retained at the site of infection (e.g., within an IAA or lesion). In some instances, the pharmaceutical compositions described herein are administered to the individual at a dosage and frequency sufficient for the concentration of CD101 at the site of infection (e.g., within an IAA or lesion) to be maintained at a concentration that is equal to or greater than the MPC for about 12 hours to about 75 hours (e.g., for about 12 hours, 14 hours, 16 hours, 18 hours, 20 hours, 22 hours, 24 hours, 26 hours, 28 hours, 30 hours, 35 hours, 40 hours, 45 hours, 50 hours, 55 hours, 60 hours, 65 hours, 70 hours, or 75 hours). In some instances, the concentration of CD101 at the site of infection (e.g., within an IAA or lesion) is maintained at a concentration that is equal to or greater than the MPC for about 72 hours

The pharmaceutical compositions described herein may be delivered as part of single dose or multi-dose treatment regimen. The dosage of the pharmaceutical composition (e.g., CD101 in salt or neutral form) of the present invention depends on factors including the route of administration and physical characteristics, e.g., age, weight, general health, of the individual. The dosage may be adapted by the physician in accordance with conventional factors such as the extent of the disease and different parameters of the individual. Typically, the amount of the pharmaceutical composition (e.g., CD101 in salt or neutral form) contained within one or more doses may be an amount that effectively reduces the risk of or treats IAC in an individual without inducing significant toxicity.

In some instances, a single dose of a pharmaceutical composition including CD101 (e.g., CD101 in salt or neutral form) is administered in an amount sufficient to treat or prevent IAC in an individual without requiring additional doses of an antifungal agent. In some instances, the single dose of CD101 (e.g., CD101 in salt or neutral form) is administered without concurrent administration of an additional antifungal agent (e.g., CD101 or another antifungal agent) within a time period (e.g., within 1 minute, 30 minutes, 1 hour, 2 hours, 12 hours, 24 hours, 2 days, 3 days, 4 days, 5 days, 6 days, or 1 week before or after administration of the single dose of CD101) that would confer therapeutic benefits (e.g., be systemically active) at the same time that CD101 is at a therapeutically effective concentration in the individual. In some instances, the single dose treatment is not combined with any other antifungal treatment within 1-21 days before or after administration. For example, a single dose of CD101 may be administered (e.g., orally, intravenously, subcutaneously, or intramuscularly) to an individual having or at risk of a fungal infection (e.g., IAC) and the single dose effectively prevents the fungal infection (e.g., IAC) without necessitating additional antifungal treatments before, during, or after the single dose treatment with CD101.

The single dose formulations can be administered to individuals in therapeutically effective amounts. In some instances, the single dose of CD101 (e.g., CD101 in salt or neutral form) can include a parenteral formulation (e.g., intravenous, intra-abdominal, subcutaneous, or intramuscular), and can be administered parenterally in dosages of about 50 mg to about 2000 mg (e.g., 75±25 mg, 100±25 mg, 150±50 mg, 200±50 mg, 250±50 mg, 300±50 mg, 350±50 mg, 400±50 mg, 450±50 mg, 500±100 mg, 600±100 mg, 700±100 mg, 800±100 mg, 900±50 mg, 1000 mg±100 mg, 1100±100 mg, 1200±100 mg, 1300±100 mg, 1400±100 mg, 1500±100 mg, 1600±100 mg, 1700±100 mg, 1800±100 mg, or 1900±100 mg). In some instances, the single dose can include an oral formulation of CD101 (e.g., CD101 in salt or neutral form), and can be administered orally in doses of about 50 mg to about 2000 mg (e.g., 75±25 mg, 100±25 mg, 150±50 mg, 200±50 mg, 250±50 mg, 300±50 mg, 350±50 mg, 400±50 mg, 500±100 mg, 600±100 mg, 700±100 mg, 800±100 mg, 900±50 mg, 1000 mg±100 mg, 1100±100 mg, 1200±100 mg, 1300±100 mg, 1400±100 mg, 1500±100 mg, 1600±100 mg, 1700±100 mg, 1800±100 mg, or 1900±100 mg).

In some instances, a pharmaceutical composition including CD101 (e.g., CD101 in salt or neutral form) is administered as part of a multi-dose regimen in an amount and frequency sufficient to treat or prevent IAC in an individual. The multi-dose regimen may include administration of two or more doses (two, three, four, five, six, seven, eight, nine, ten or more than ten doses) of a pharmaceutical composition including CD101 (e.g., CD101 in salt or neutral form).

In some instances, the multi-dose regimen can include a parenteral (e.g., intravenous, intra-abdominal, or subcutaneous) formulation of CD101 (e.g., CD101 in salt or neutral form). For example, CD101 (e.g., CD101 in salt or neutral form) can be parenterally (e.g., intravenously, intra-abdominally intramuscularly, or subcutaneously) administered in dosages of about 50-1200 mg (e.g., 50-125 mg, 75-150 mg, 100-175 mg, 125-200 mg, 150-225 mg, 175-250 mg, 200-275 mg, 225-300 mg, 250-325 mg, 275-350 mg, 300-375 mg, 325-400 mg, 350-425 mg, 375-450 mg, 400-475 mg, 425-500 mg, 450-525 mg, 475-550 mg, 500-575 mg, 525-600 mg, 550-625 mg, 575-650 mg, 600-675 mg, 625-700 mg, 650-725 mg, 675-750 mg, 700-775 mg, 725-800 mg, 750-825 mg, 775-850 mg, 800-875 mg, 825-900 mg, 850-925 mg, 875-950 mg, 900-975 mg, 925-1000 mg, 950-1025 mg, 975-1050 mg, 1000-1075 mg, 1025-1100 mg, 1050-1125 mg, 1075-1150 mg, 1100-1175 mg, 1125-1200 mg, 50-250 mg, 250-400 mg, 200-600 mg, 400-1200 mg, 50-400 mg, or 50-1200 mg). In some instances, the first dose contains about 400 mg of CD101, or a neutral form thereof and each of the subsequent doses contains about 200 mg of a salt of CD101, or a neutral form thereof. In some instances, the first dose includes about 400 mg of CD101, or a salt or neutral form thereof, and each of the subsequent doses include about 50-400 mg (e.g., 50-125 mg, 75-150 mg, 100-175 mg, 125-200 mg, 150-225 mg, 175-250 mg, 200-275 mg, 225-300 mg, 250-325 mg, 275-350 mg, 300-375 mg, 325-400 mg, 50-250 mg, 250-400 mg, or 50-400 mg) of CD101, or a salt or neutral form thereof. The parenteral formulation can be administered once or multiple times at regular (e.g., daily, every two days, every three days, weekly) or irregular intervals. In some instances, the parenteral formulation is administered in combination with an oral dosage formulation. The administration of the parenteral dosage form can coincide or occur at different times (e.g., 15 minutes, 45 minutes, 1 hour, 12 hours, 24 hours, or 3 days) relative to the time of administration of the oral dosage form. In instances, the parenteral dosage form can be administered to an individual in one or more weekly doses (e.g., 1, 2, 3, or 4 doses/month) or one or more monthly doses (1, 2, 3, 4, 5, 6, 7, 8, 9, 10, 11, 12 doses/year). Alternatively, the parenteral dosage form can be administered once to an individual, with no additional dosages later on.

In some instances, the multi-dose treatment regimen can include an oral formulation of CD101 (e.g., CD101 in salt or neutral form), and can be administered orally in doses of about 50 mg to about 2000 mg (e.g., 75±25 mg, 100±25 mg, 150±50 mg, 200±50 mg, 250±50 mg, 300±50 mg, 350±50 mg, 400±50 mg, 500±100 mg, 600±100 mg, 700±100 mg, 800±100 mg, 900±50 mg, 1000 mg±100 mg, 1100±100 mg, 1200±100 mg, 1300±100 mg, 1400±100 mg, 1500±100 mg, 1600±100 mg, 1700±100 mg, 1800±100 mg, or 1900±100 mg). The oral dosage of CD101 (e.g., CD101 in salt or neutral form) or any formulation of an antifungal agent can be administered daily or one or more times per week (e.g., 1, 2, 3, 4, 5, or 6 days a week). For example, the dosage can be administered one or more times per week over the course of 2 to 8 weeks (e.g., 2 to 8 weeks, 2 to 7 weeks, 2 to 6 weeks, 2 to 5 weeks, 2 to 4 weeks, or 2 to 3 weeks).

In a multi-dose treatment regimen, the timing of the administration of a compound of each dosage (e.g., CD101 in salt or neutral form thereof) depends on the medical and health status of the individual. The timing of the administration of each dosage (e.g., CD101 in salt or neutral form) may be optimized by a physician to treat or reduce the likelihood of a fungal infection (e.g., IAC) in an individual.

In any of the methods described herein, the pharmaceutical composition can be administered in a dosage pattern, frequency, or duration to effectively reduce the likelihood of a fungal infection (e.g., IAC) in an individual. In some instances, the pharmaceutical composition is administered one or more times per year (e.g., 1, 2, 3, 4, 5, or 6 times per year), one or more times per month (e.g., 1, 2, 3, or 4 times per month), one or more times per week (e.g., 1, 2, 3, 4, 5, 6, or 7 times per week), or one or more times per day (e.g., 1, 2, or 3 times per day). In some instances, the pharmaceutical composition is administered on consecutive days (e.g., every day), consecutive weeks (e.g., every week), or consecutive months (e.g., every month). In some instances, the pharmaceutical composition is administered on non-consecutive days (e.g., every other day, every 3 days, every 4 days, every 5 days, or every 6 days), weeks (e.g., every other week or every 2 or 3 weeks), or months (e.g., every other month or every 2, 3, 4, 5, 6, 7, 8, 9, 10, or 11 months). In some instances, the pharmaceutical composition is administered for a duration of about 1 to 8 weeks (e.g., 1 to 3, 2 to 4, 3 to 5, 4 to 6, 5 to 7, or 6 to 8 weeks). In some instances, the pharmaceutical composition is administered for a duration of about 2 to 12 months (e.g., 2 to 4, 3 to 5, 4 to 6, 5 to 7, 6 to 8, 7 to 9, 8 to 10, 9 to 11, or 10 to 12 months).

IV. Combination Therapies

In some instances, the pharmaceutical composition including CD101 in salt or neutral form may be administration in combination with one or more additional anti-microbial agents (e.g., antifungal agent or antibacterial agent) to treat or prevent IAC associated with a co-infection, wherein the co-infection involves Candida and one or more additional fungal or bacterial strains. Antifungal agents and antibacterial agents that may be administered in combination with CD101 are further described below.

Antifungal Agents

In the multi-dose regimens described herein, a second antifungal agent can be used in combination with a pharmaceutical composition including CD101 (e.g., CD101 in salt or neutral form) to treat, reduce the likelihood of, or prevent, a fungal infection (e.g., IAC). The second antifungal agent and the pharmaceutical composition including CD101 (e.g., CD101 in salt or neutral form) can be administered concurrently. Alternatively, the pharmaceutical composition including CD101 (e.g., CD101 in salt or neutral form) can be administered first, followed by administration of the second antifungal agent. In some instances, the second antifungal agent is administered first, followed by administration of the pharmaceutical composition including CD101 (e.g., CD101 in salt or neutral form).

Antifungal agents that can be used as a second antifungal agent in combination with a pharmaceutical composition including CD101 (e.g., CD101 in salt or neutral form) include, but are not limited to, CD101 (e.g., a salt of CD101 or neutral form thereof), clindamycin (sold under the brand names CLEOCIN® and DALACIN®), trimethoprim (sold under the brand names PROLOPRIM®, MONOTRIM®, and TRIPRIM®), sulfamethoxazole (sold under the brand name GANTANOL®), cotrimoxazole (a combination of trimethoprim and sulfamethoxazole (aka TMP-SMX); this combination is sold under the brand names BACTRIM®, COTRIM®, SULFATRIM®, and SEPTRA®), atovaquone (sold under the brand name MEPRON®), pentamidine (sold under the brand names NEBUPENT® and PENTRAM®), primaquine, pyrimethamine (sold under the brand name DARAPRIM®), and pharmaceutically acceptable salts thereof.

Alternatively, the second antifungal agent described herein can be selected from glucan synthase inhibitors (e.g., echinocandins, enfumafungins), polyene compounds, azole compounds, and pharmaceutically acceptable salts thereof.

Glucan synthase inhibitors that can be used as a second antifungal agent include, but are not limited to echinocandins (e.g., caspofungin, micafungin, or anidulafungin) enfumafungin (e.g., SCY-078 (aka MK-3118, see Lepak et al., Antimicrobial agents and chemotherapy 59:1265 (2015)), and pharmaceutically acceptable salts thereof.

The azole compounds are antifungal compounds that contain an azole group (i.e., a five-membered heterocyclic ring having at least one N and one or more heteroatoms selected from N, O, or S). Azole compounds function by binding to the enzyme 14α-demethylase and disrupt, inhibit, and/or prevent its natural function. The enzyme 14α-demethylase is a cytochrome P450 enzyme that catalyzes the removal of the C-14 α-methyl group from lanosterol before lanosterol is converted to ergosterol, an essential component in the fungal cell wall. Therefore, by inhibiting 14α-demethylase, the synthesis of ergosterol is inhibited. Azole compounds that can be used in the first dosage form of the invention include, but are not limited to (e.g., VT-1161, VT-1129, VT-1598, fluconazole, albaconazole, bifonazole, butoconazole, clotrimazole, econazole, efinaconazole, fenticonazole, isavuconazole, isoconazole, itraconazole, ketoconazole, luliconazole, miconazole, omoconazole, oxiconazole, posaconazole, pramiconazole, ravuconazole, sertaconazole, sulconazole, terconazole, tioconazole, and voriconazole), VL-2397, and flucytosine (ANCOBON®).

Polyene compounds are compounds that insert into fungal membranes, bind to ergosterol and structurally related sterols in the fungal membrane, and disrupt membrane structure integrity, thus causing leakage of cellular components from a fungus that causes infection. Polyene compounds typically include large lactone rings with three to eight conjugated carbon-carbon double bonds and may also contain a sugar moiety and an aromatic moiety. Polyene compounds typically include large lactone rings with three to eight conjugated carbon-carbon double bonds and may also contain a sugar moiety and an aromatic moiety. Polyene compounds that can be used in the first dosage form of the invention include, but are not limited to, 67-121-A, 67-121-C, amphotericin B, derivatives of amphotericin B (e.g., C35deOAmB; see Gray et al., Proceedings of the National Academy of Sciences 109:2234 (2012)), arenomvcin B, aurenin, aureofungin A, aureotuscin, candidin, chinin, chitin synthesis inhibitors (e.g., lufenuron), demethoxyrapamycin, dermostatin A, dermostatin B, DJ-400-B1, DJ-400-B2, elizabethin, eurocidin A, eurocidin B, filipin I, filipin II, filipin III, filipin IV, fungichromin, gannibamycin, hamycin, levorin A2, lienomycin, lucensomycin, mycoheptin, mycoticin A, mycoticin B, natamycin, nystatin A, nystatin A3, partricin A, partricin B, perimycin A, pimaricin, polifungin B, rapamycin, rectilavendomvcin, rimocidin, roflamycoin, tetramycin A, tetramycin B, tetrin A, tetrin B, and pharmaceutically acceptable salts thereof.

Other compounds that have antifungal properties that may be used as the second antifungal agent include, but are not limited to polygodial, benzoic acid, ciclopirox, tolnaftate, undecylenic acid, flucytosine or 5-fluorocytosine, griseofulvin, and haloprogin.

Antibacterial Agents

In the multi-dose regimens described herein, an antibacterial agent can be used in combination with a pharmaceutical composition including CD101 (e.g., CD101 in salt or neutral form) to treat, reduce the likelihood of, or prevent, IAC associated with a co-infection. In some instances, the antibacterial agent and the pharmaceutical composition including CD101 (e.g., CD101 in salt or neutral form) can be administered concurrently. In some instances, the pharmaceutical composition including CD101 (e.g., CD101 in salt or neutral form) can be administered first, followed by administration of the antibacterial agent. In some instances, the antibacterial agent is administered first, followed by administration of the pharmaceutical composition including CD101 (e.g., CD101 in salt or neutral form).

Antibacterial agents include, but are not limited to, aminoglycosides (e.g., amikacin (AMIKIN®), gentamicin (GARAMYCIN®), kanamycin (KANTREX®), neomycin (MYCIFRADIN®), netilmicin (NETROMYCIN®), tobramycin (NEBCIN®), Paromomycin (HUMATIN®)), ansamycins (e.g., geldanamycin, herbimycin), carbacephem (e.g., loracarbef (LORABID®), Carbapenems (e.g., ertapenem (INVANZ®), doripenem (DORIBAX®), imipenem/cilastatin (PRIMAXIN®), meropenem (MERREM®), cephalosporins (first generation) (e.g., cefadroxil (DURICEF®), cefazolin (ANCEF®), cefalotin or cefalothin (KEFLIN®), cefalexin (KEFLEX®), cephalosporins (second generation) (e.g., cefaclor (CECLOR®), cefamandole (MANDOL®), cefoxitin (MEFOXIN®), cefprozil (CEFZIL®), cefuroxime (CEFTIN®, ZINNAT®)), cephalosporins (third generation) (e.g., cefixime (SUPRAX®), cefdinir (OMNICEF®, CEFDIEL®), cefditoren (SPECTRACEF®), cefoperazone (CEFOBID®), cefotaxime (CLAFORAN®), cefpodoxime (VANTIN®), ceftazidime (FORTAZ®), ceftibuten (CEDAX®), ceftizoxime (CEFIZOX®), ceftriaxone (ROCEPHIN®)), cephalosporins (fourth generation) (e.g., cefepime (MAXIPIME®)), cephalosporins (fifth generation) (e.g., ceftobiprole (ZEFTERA®)), glycopeptides (e.g., teicoplanin (TARGOCID®), vancomycin (VANCOCIN®), telavancin (VIBATIV®)), lincosamides (e.g., clindamycin (CLEOCIN®), lincomycin (LINCOCIN®)), lipopeptide (e.g., daptomycin (CUBICIN®)), macrolides (e.g., azithromycin (ZITHROMAX®, SUMAMED®, ZITROCIN®), clarithromycin (BIAXIN®), dirithromycin (DYNABAC®), erythromycin (ERYTHOCIN®, ERYTHROPED®), roxithromycin, troleandomycin (TAO®), telithromycin (KETEK®), spectinomycin (TROBICIN®)), monobactams (e.g., aztreonam (AZACTAM®)), nitrofurans (e.g., furazolidone (FUROXONE®), nitrofurantoin (MACRODANTIN®, MACROBID®)), penicillins (e.g., amoxicillin (NOVAMOX®, AMOXIL®), ampicillin (PRINCIPEN®), azlocillin, carbenicillin (GEOCILLIN®), cloxacillin (TEGOPEN®), dicloxacillin (DYNAPEN®), flucloxacillin (FLOXAPEN®), mezlocillin (MEZLIN®), methicillin (STAPHCILLIN®), nafcillin (UNIPEN®), oxacillin (PROSTAPHLIN®), penicillin G (PENTIDS®), penicillin V (PEN-VEE-K®), piperacillin (PIPRACIL®), temocillin (NEGABAN®), ticarcillin (TICAR®)), penicillin combinations (e.g., amoxicillin/clavulanate (AUGMENTIN®), ampicillin/sulbactam (UNASYN®), piperacillin/tazobactam (ZOSYN®), ticarcillin/clavulanate (TIMENTIN®)), polypeptides (e.g., bacitracin, colistin (COLY-MYCIN-S®), polymyxin B, quinolones (e.g., ciprofloxacin (CIPRO®, CIPROXIN®, CIPROBAY®), enoxacin (PENETREX®), gatifloxacin (TEQUIN®), levofloxacin (LEVAQUIN®), lomefloxacin (MAXAQUIN®), moxifloxacin (AVELOX®), nalidixic acid (NEGGRAM®), norfloxacin (NOROXIN®), ofloxacin (FLOXIN®, OCUFLOX®), trovafloxacin (TROVAN®), grepafloxacin (RAXAR®), sparfloxacin (ZAGAM®), temafloxacin (OMNIFLOX®)), sulfonamides (e.g., mafenide (SULFAMYLON®), sulfonamidochrysoidine (PRONTOSIL®), sulfacetamide (SULAMYD®, BLEPH-10®), sulfadiazine (MICRO-SULFON®), silver sulfadiazine (SILVADENE®), sulfamethizole (THIOSULFIL FORTE®), sulfamethoxazole (GANTANOL®), sulfanilimide, sulfasalazine (AZULFIDINE®), sulfisoxazole (GANTRISIN®), trimethoprim (PROLOPRIM®), TRIMPEX®), trimethoprim-sulfamethoxazole (co-trimoxazole) (TMP-SMX) (BACTRIM®, SEPTRA®)), tetracyclines (e.g., demeclocycline (DECLOMYCIN®), doxycycline (VIBRAMYCIN®), minocycline (MINOCIN®), oxytetracycline (TERRAMYCIN®), tetracycline (SUMYCIN®, ACHROMYCIN® V, STECLIN®)), drugs against mycobacteria (e.g., clofazimine (LAMPRENE®), dapsone (AVLOSULFON®), capreomycin (CAPASTAT®), cycloserine (SEROMYCIN®), ethambutol (MYAMBUTOL®), ethionamide (TRECATOR®), isoniazid (I.N.H.®), pyrazinamide (ALDINAMIDE®), rifampin (RIFADIN®, RIMACTANE®), rifabutin (MYCOBUTIN®), rifapentine (PRIFTIN®), streptomycin), and others (e.g., arsphenamine (SALVARSAN®), chloramphenicol (CHLOROMYCETIN®), fosfomycin (MONUROL®), fusidic acid (FUCIDIN®), linezolid (ZYVOX®), metronidazole (FLAGYL®), mupirocin (BACTROBAN®), platensimycin, quinupristin/dalfopristin (SYNERCID®), rifaximin (XIFAXAN®), thiamphenicol, tigecycline (TIGACYL®), tinidazole (TINDAMAX®, FASIGYN®)).

The following examples are intended to further illustrate but not to limit the invention herein.

### EXAMPLES

### Example 1. Drug Penetration of Echinocandin Antifungals at the Site of Infection in an Intra-Abdominal Abscess Model

The objective of this study was to critically evaluate the penetration of echinocandin drugs at the site of infection and assess whether drug levels in lesions help account for the observed clinical response and potential for later stage resistance emergence.

Experimental Method

Candida albicans Strain and Antifungal Drugs.

C. albicans strain SC4315 was grown in yeast extract peptone dextrose broth at 37° C. with shaking overnight. Cells were washed, counted, and prepared to 1×10⁸ colony forming units (CFU)/ml for inoculation. CD101, CD101-D9 (Cidara Therapeutics, Inc., San Diego, Calif., USA), and micafungin (Astellas Pharma Inc., Tokyo, Japan) were obtained as standard powders from their manufacturer. ¹³C₆-micafungin was purchased from ALSACHIM, France.

Mouse Model of Intra-Abdominal Candidiasis and Tissue Sample Collection.

A mouse model of IAC was used for this study. Female six to eight week old CD1 mice (Charles River Laboratories) weighing 18-22 g were infected intraperitoneally (IP) with 1×10⁷ CFU of C. albicans SC5314 mixed with sterile stool matrix as previously described. Single IP doses of CD101 at 20 mg/kg (equivalent to humanized therapeutic dose) or micafungin at 5 mg/kg (therapeutic dose) were administered to groups of 15 mice at day three post-inoculation. Mice were sacrificed just before antifungal treatment (n=1), and at 1, 3, 6, 24, and 48 hours post-dose (three mice per group per time point). Livers and kidneys were explored for abscesses >1 mm in diameter, dissected, placed on a cryohistology tray, and snap-frozen in liquid nitrogen and stored at −80° C. for tissue sectioning for MALDI imaging.

In a further experiment, a low dose of CD101 at 5 mg/kg was administered and micafungin was given at the same dose. Liver and kidney samples were collected at 6 hours and 24 hours post-dose for both MALDI imaging and absolute drug quantification.

In another separate experiment that aimed at comparing therapeutic level of single dose CD101 and multiple doses of micafungin, a single dose of CD101 at 20 mg/kg was given at day three post-inoculation, and once daily treatment of micafungin at 5 mg/kg starting from day three post-inoculation and a total of three doses of micafungin was administered. Livers and kidneys were collected at 48 hours and 72 hours post first dose of each drug.

Tissue Sectioning and Matrix Application.

Tissues were sectioned at 12 μm thickness using a Leica CM1850 cryostat (Buffalo Grove, Ill.) and mounted onto stainless steel slides (for MALDI-MSI analysis) or frosted glass microscope slides (for H&E staining). Tissue sections were stored at −80° C. until analysis. Prior to MALDI-MSI analysis, tissue sections were thawed and applied with ionization matrix. For CD101, 2,5-dihydroxybenzoic acid (DHB) (20 mg/ml in 50% methanol) containing 267 fmol/μl CD101-D9 (Cidara Therapeutics, Inc) was applied to the surface using a HTX TM sprayer (Chapel Hill, N.C.) operating with 50 μL/min flow rate, 60° C. nozzle temperature and 5 p.s.i. Twenty-five passes over the tissue were performed. For micafungin, 1,5-diaminonaphthalene (1,5-DAN) matrix (5 mg/ml in 50% acetone) containing 5 nmol/μl ¹³C₆-micafungin (Alsachim, France) was coated onto tissue surface by the TM-sprayer at flow rate of 60 μl/min, 50° C. nozzle temperature and 5 p.s.i. Twenty-five passes over the tissue were performed.

MALDI-MSI Analysis.

MALDI-MSI analysis was performed using a MALDI LTQ Orbitrap XL mass spectrometer (Thermo Fisher Scientific, Bremen, Germany) with a resolution of 60,000 at m/z 400, full width at half maximum. The resolution was sufficient to resolve CD101, CD101-D9, micafungin, and ¹³C₆-micafungin peaks from background without the requirement from MS/MS and subsequent loss of signal. However, drug peak identities were confirmed by acquiring several MS/MS spectra directly from the dosed tissues. Standards of CD101 and micafungin were analyzed both direct from the stainless steel target plate and spiked into drug-naïve liver tissue to optimize instrument parameters. Limit of detection (LOD) for MALDI-MSI analysis of micafungin and CD101 was 500 ng/g and 1 μg/g of liver or kidney tissue respectively, calculated as described.

For CD101, spectra were acquired in the m/z 1000-1500 range, using positive ionization with a laser energy of 25 μJ and 15 laser shots were fired at each position. Spectra for micafungin were acquired in the same m/z range under negative ionization mode with a laser energy of 10 μJ and 5 laser shots at each position. The laser step size was set at between 50-75 μm, at which small necrotic areas within lesions could easily be resolved and no overlapping of the laser spot on adjacent acquisitions was observed.

Data visualization was performed using Thermo ImageQuest software. Normalized ion images of CD101 were generated by dividing CD101 [M+H]⁻ signal (m/z 1225.603±0.005) by CD101-D9 [M+H]⁺ signal (m/z 1234.651±0.005). Normalized ion images of micafungin [M−H]⁻ signal (m/z 1268.444±0.005) by ¹³C₆-micafungin [M−H]⁻ signal (m/z 1274.455±0.005).

Laser-capture microdissection. Necrotic lesion and surrounding tissue areas totaling two to six million m² were dissected from between three and six serial liver or kidney biopsy tissue sections using a Leica LMD6500 system (Buffalo Grove, Ill.). Lesion areas were identified optically from the brightfield image scan and by comparison to the adjacently-sectioned H&E reference tissue. Pooled dissected lesion tissues were collected into 0.25 ml standard PCR tubes and immediately transferred to the −80° C. freezer for storage.

Prior to analysis, the tubes were thawed at room temperature for 30 minutes. 50 μl of extraction solution (ACN/MeOH (1/1) with 100 ng/ml CD101-D9 and 100 ng/ml ¹³C₆-micafungin) was added to each tube, which were then sonicated for five min and centrifuged at 10000 RPM for five min at room temperature. 40 μl of supernatant was transferred for LC/MS-MS analysis and diluted with an additional 40 μl of MilliQ water.

Neat 1 mg/ml DMSO stocks for all compounds were serial diluted in 50/50 acetonitrile water to create standard curves and quality control spiking solutions. 3 μl of neat spiking solutions were added to 2 μl of lesion homogenate and extraction was performed by adding 50 μl of extraction solution (ACN/MeOH (1/1) with 100 ng/ml CD101-D9 and 100 ng/ml ¹³C₆-micafungin). Extracts were vortexed for five minutes and centrifuged at 10000 RPM for five min. A 40 μl of supernatant was transferred for LC/MS-MS analysis and diluted with an additional 40 μl of MilliQ water. Previously optimized LC/MS-MS parameters were used for analysis (see LC/MS-MS section).

Drug Quantitation by LC/MS-MS.

LC-MS analysis was performed on a Q Exactive high resolution mass spectrometer (Thermo Fisher Scientific, Waltham, Mass.) coupled to a Thermo Scientific Dionex UltiMate 3000 binary system. Chromatography was performed with a Kinetex C18 column (2.1×50 mm; particle size 1.7 μm, Phenomenex, Torrance, Calif.) using a reverse phase gradient elution, ACN:H₂O (60:40) and 10 mM ammonium acetate for mobile phase A and IPA:ACN:MeOH (80:10:10) and 10 mM ammonium acetate for mobile phase B. A flow rate of 300 μl/min was used, with a gradient consisting of 20% B held for 0.5 min, followed by linear increase to 95% in 3.5 min, held for 2.2 min and return to the initial 20% B in 0.3 min. The column was equilibrated for 1.5 min before the next injection, and the temperature of column and sample tray were held at 50 and 4° C., respectively. The column retention time for CD101 and micafungin was 3.32 min and 3.2 min respectively.

Key MS parameters were as follow: the spray voltage, 3.5 kV; capillary temperature, 320° C.; HESI probe temperature, 400° C.; S-lens RF level, 50. The sheath gas and auxiliary gas were set to 45 and 10 units, respectively. External mass calibration was performed before each sequence. For CD101, full scan was applied in positive ionization mode with a mass range of m/z 250-1500 at resolution power 70,000, AGC target 3e6 for a maximum IT of 100 ms. For micafungin, full scan was applied in negative ionization mode with a mass range of m/z 250-1500 at resolution power 70,000, AGC target 3e6 for a maximum IT of 100 ms. CD101 [M+H]+ signal was normalized to CD101-D9 [M+H]+ and micafungin [M+H]− signal was normalized to ¹³C₆-micafungin [M+H]−.

Statistical Analysis.

Absolute drug concentrations were graphed and statistically analyzed in the GraphPad software (Prism 7; GraphPad Software, Inc., San Diego, Calif.). Drug levels in different tissue compartment at different time points were compared by the one way analysis of variance (ANOVA) and Dunn’ multiple comparison was used for the post hoc analyses. Statistical significance was defined as P<0.05. Prompt antifungal therapy and source controls are crucial for successful treatment.

Results

Tissue Distribution and Penetration after a Single Dose of Micafungin and CD101.

Histopathology and MALDI Imaging Analysis.

The IAC model yielded abundant heterogeneous lesions at three days post infection. Echinocandin antifungal drugs were then introduced and the spatial distribution of micafungin and CD101 was visualized by MALDI mass spectrometry imaging (MALDI-MSI), delivering high-resolution heat maps of drug concentration in liver and kidney tissues. Side by side comparison of these images with histopathological staining (Hematoxylin and Eosin (H&E) and Gomori Methenamine Silver (GMS)) of adjacent sections revealed drug penetration in different lesion structures, as well as the relationship between drug distribution and location of fungal cells. Upon histopathological analysis, it was observed that lesions formed in abdominal organs are characterized by large macrophage/neutrophil infiltrates surrounding a necrotic core of various sizes. Fungal-specific GMS staining further supported that fungal load in lesions appeared to correlate with necrotic severity of lesion. Predominantly, high fungal staining was observed in necrotic area.

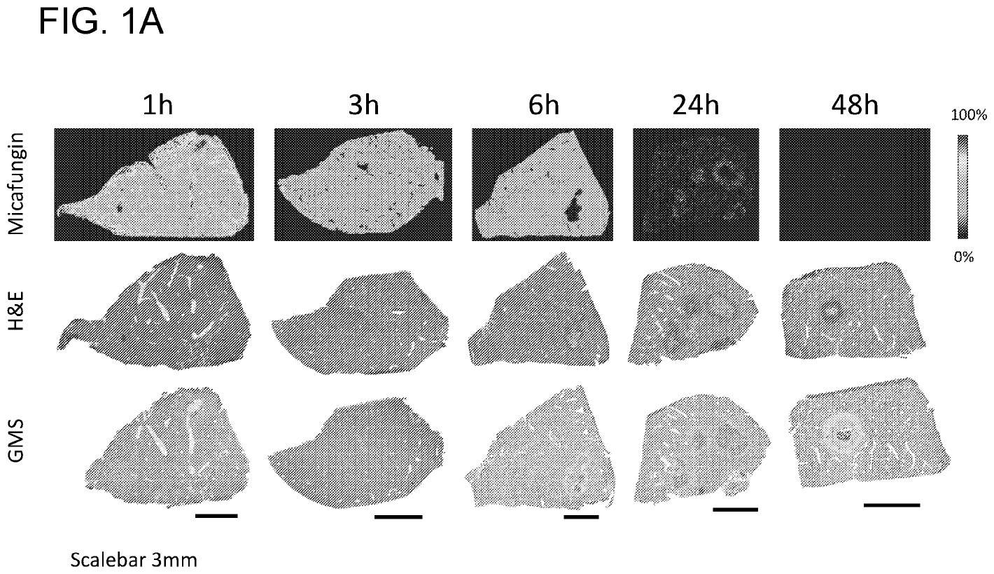

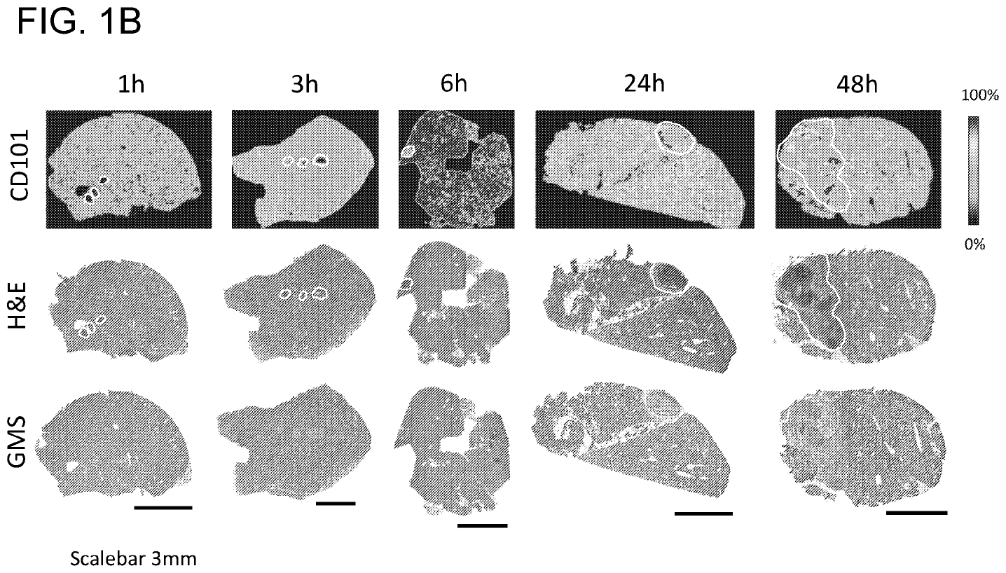

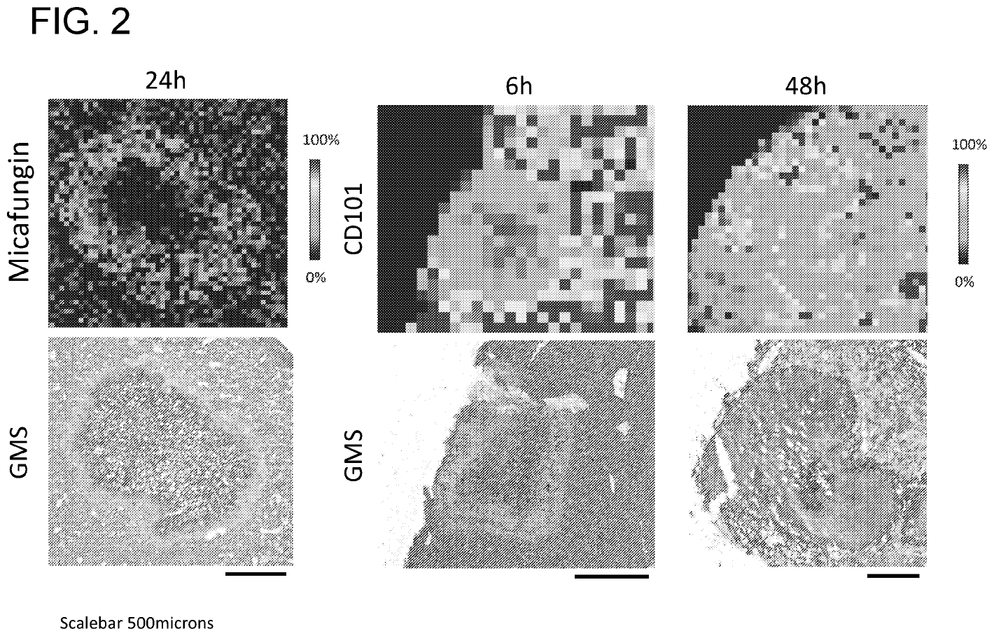

After a single humanized dose, both echinocandins were quickly distributed into liver and kidney. However, the pharmacokinetics of tissue exposure and the pattern of lesion penetration were notably different for these two drugs. After a single dose administration of micafungin at 5 mg/kg, drug quickly distributed into liver tissues and reached peak intensity at 1 h (FIG. 1A). Decreased drug intensities (relative drug abundance) over the entire tissue were observed at 3 hours and 6 hours; although, drug signal was barely detectable in lesions until 6 hours when the drug was observed at the edge of the lesion with little detected penetration into the necrotic core. Penetration of the drug into the necrotic lesion was clearly observed at 24 hours, when the micafungin signal was detected inside of the lesion with noticeably higher intensities in the outer rim of the lesion relative to the necrotic center, as well as the surrounding uninvolved tissue. An enlarged view of the 24-hour MALDI image and the adjacent GMS stained section (FIG. 2) showed that micafungin predominantly resided in the lesion edge, whereas fungal cells constitute a massive network throughout the entire lesion. Thus, interaction between the drug and fungi was limited in the outer part of the lesion. But within the necrotic center where the majority of the fungal population reside, there was no detectable drug exposure (Limit of detection for MALDI-MSI analysis of Micafungin and CD101 was 500 ng/g and 1 μg/g of liver or kidney tissue respectively). Moreover, without further dosing past 24 hours, such drug retention within the lesion was too low to outcompete the quick drug clearance from liver, where drug levels dropped far below the limit of detection and no drug signals were detected at 48 hours. In kidneys, the same kinetic pattern of tissue distribution and lesion penetration was observed for micafungin.

In liver, CD101 signal intensity was readily detected at the earliest time point investigated (one hour post-dose) and steadily increased at 3 hours and 6 hours, which maxed out at 6 hours post-dose (FIG. 1B). Thereafter, CD101 drug intensity slowly declined but still persisted strongly even at 48 hours post-dose. Closer examination of the ion map and GMS staining revealed detectable lesion penetration (CD101 signal appeared inside of lesion) as early as 3 hours and a gradient of drug distribution was observed within the lesion at 6 hours with higher drug intensity in the outer area and less signals in the necrotic center (FIG. 2). At later time points (24 hours and 48 hours), CD101 was primarily persisting with slow accumulation within the lesion while surrounding tissue drug levels were declining. The 48-hour enlarged view mapped out a more homogeneous distribution of CD101 within the lesion (FIG. 2). Drug distribution and penetration of CD101 in kidneys were similar to what was observed in liver, whereas lesion formation and histopathology in kidneys was not as consistent as what was observed in livers with heterogeneous manifestations ranging from very tiny lesions to big or multifocal lesions.

Quantitative Evaluation of Drug Exposure in Liver Lesions.

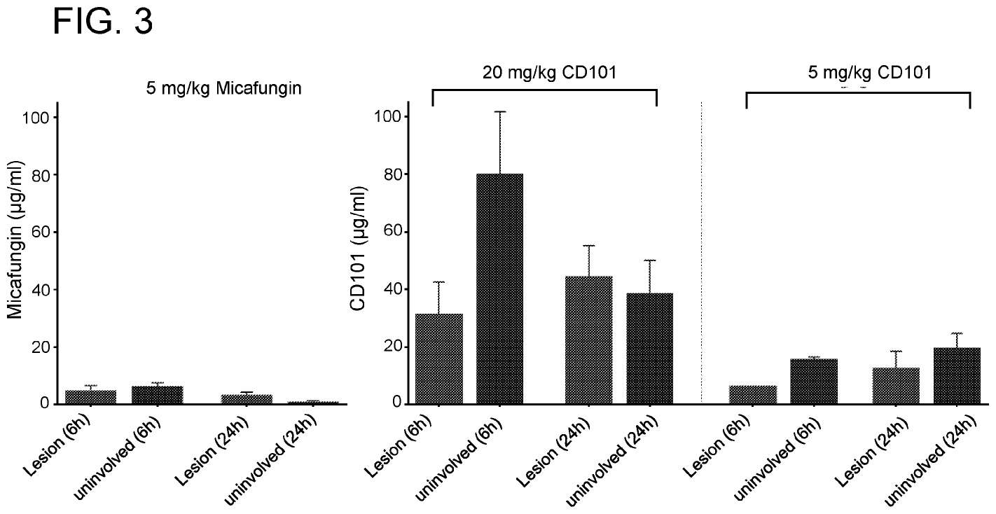

MALDI imaging analysis provides valuable information on spatial distribution and lesion penetration. Yet, it is only semi-quantitative and not a measure of exact drug exposure at the site of infection. Hence, laser capture microdissection (LCM) was next applied, followed by high-pressure liquid chromatography coupled tandem mass spectrometry (LC/MS-MS) to quantify absolute drug concentration in distinct compartments of involved tissues at two representative time points, 6 hours and 24 hours post-dose. Only liver samples were analyzed due to the fact that kidney lesions were too small to meet the minimal quantification requirement. Upon quantification (FIG. 3), the single dose of 5 mg/kg micafungin (at which dosage, experimental serum drug levels ranged approximately from 7 to 10 μg/ml at 6 hours and around 2 μg/ml at 24 hours resulted in drug retention at 6.5 and 1 μg/ml in uninvolved surrounding tissues and 4.9 and 3.4 μg/ml in lesions at 6 hours and 24 hours, respectively. In contrast, remarkably high levels of CD101 were found in liver tissues. After therapeutic dosing (20 mg/kg) of CD101 (which resulted in serum drug levels of 43 and 22 μg/ml at 6 hours and 24 hours, respectively, the average drug level at 6 hours was 80.1 μg/ml in non-lesion part and 31.6 μg/ml in lesions. At 24 hours, the drug level in surrounding tissue dropped significantly (P=0.01) but were still high with a mean concentration of 38.7 μg/ml. Moreover, the mean drug concentration within lesions increased to 44.5 μg/ml at 24 hours even though the statistical significance of such increase was not achieved due to the small sample size. When the lower dose CD101 at 5 mg/kg was administered, a proportionally decreased but high level of drug was observed from both compartments at both time points. Drug concentration was 15.8 μg/ml at 6 hours and 19.8 μg/ml at 24 hours in surrounding tissues, and 6.6 and 12.7 μg/ml in lesions at 6 hours and 24 hours, respectively.

Comparison of Drug Accumulation at Site of Infection after Multiple Doses of Micafungin and Single Dose of CD101.

Maldi Imaging.

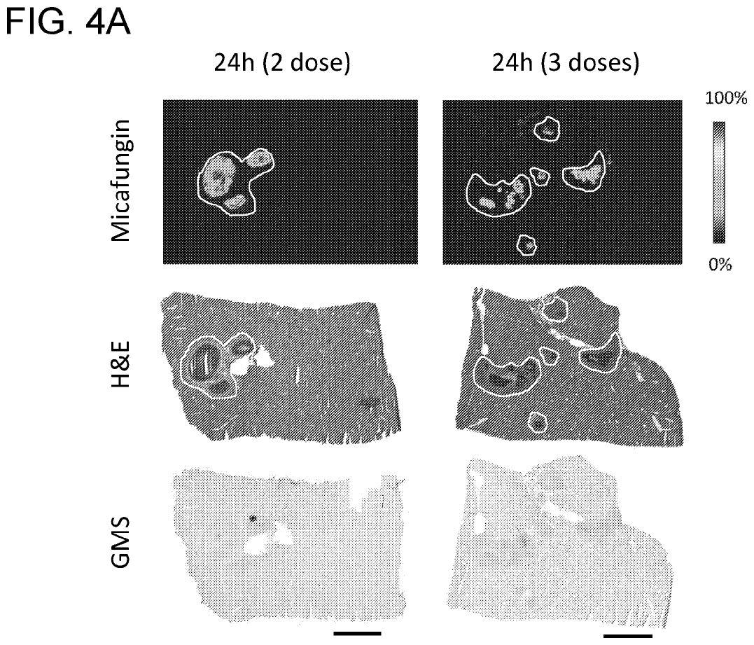

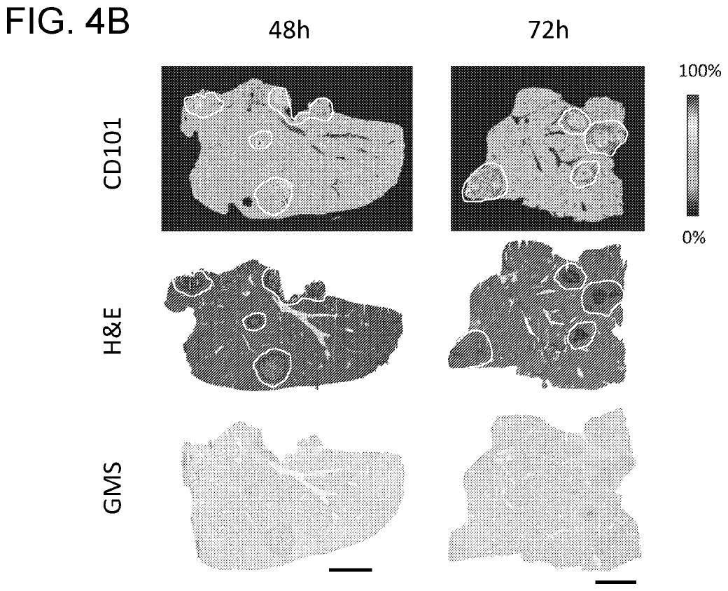

Given the fact that the standard micafungin regimen in the clinical setting is daily dosing, a multiple-dose experiment was designed to look into drug accumulation of micafungin at steady-state. Drug distribution at 24 hours following two and three therapeutic doses of micafungin was analyzed. MALDI imaging analysis (FIG. 4A) showed that multi-dosing had limited impact on partitioning into liver tissue, as drug signals were barely captured in the non-lesion part of tissues even after three doses of micafungin. In contrast to marginally detectable drug levels in the surrounding tissues, a noticeably increased drug intensity was observed inside lesions after multiple doses, indicating micafungin was accumulating somewhat within lesion at above normal tissue drug level at the steady state. A single therapeutic dose CD101 arm was established in parallel with the multi-dosing micafungin arm. Tissue samples from the CD101 arm were collected at 48 hours and 72 hours post-dose, equivalent to 24 hours post two and three doses of micafungin, respectively. Consistent with the previous single dose experiment, at 48 hours, robust CD101 signal was detected from the entire tissue and drug diffused into lesions more effectively (FIG. 4B). Drug accumulation within the necrotic region of the lesions became more visible at 72 hours, when drug intensities in the surrounding tissue were reduced.

Quantifying Drug Levels.

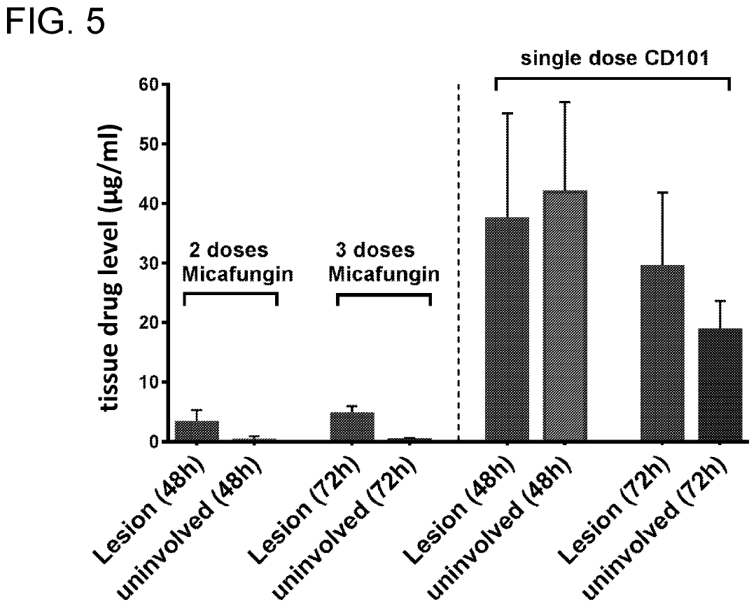

Compared to single dose micafungin, additional daily dosing, which reached steady-state after three doses, promoted drug retention within lesion even though drug level in surrounding non-lesion tissues was low at only 0.5 μg/ml and not much different from that at 24 hours after single dosing (FIG. 5). Micafungin accumulated in lesions slowly but continuously, retaining 3.5 μg/ml and 4.9 μg/ml at 24 hours post second and third drug dose, respectively. In comparison, the extensive tissue distribution and lesion penetration after single dose CD101 was confirmed once again when samples were assessed at an extended time point to match steady-state micafungin sample collection. A mean CD101 concentration of 37.7 μg/ml and 29.7 μg/ml was reported from dissected lesions at 48 hours and 72 hours, respectively, and corresponding drug levels in surrounding tissues were measured at 42.2 μg/ml and 19.1 μg/ml (FIG. 5). These results are consistent with the MALDI imaging data.

Conclusions

Herein, by employing MALDI imaging technology and LCM-directed drug quantification in a clinically relevant IAC mouse model, drug exposure within intra-abdominal abscesses has been assessed.

IAC is difficult to treat and outcome is poor even after proper source control and adequate antifungal treatment. These observations have raised concern about insufficient drug penetration during therapy for IAC. Interestingly, the data revealed that micafungin was gradually penetrating into liver and kidney abscesses, but only reached detectable levels inside lesions at 6 hours after the first dose. The penetration improved upon multiple doses of treatment, and only at steady-state were drug signals observed from the necrotic core where large amount of fungal cells proliferate. Absolute drug quantification from lesions at different post-dose time points further confirmed this drug accumulation pattern.

In the present study, CD101 has an extensive tissue distribution with an impressive drug level of 80.1 μg/ml in non-lesion part of liver at 6 hours after a single dose treatment at 20 mg/kg. More notably, the drug was observed to quickly penetrate into abscesses as early as 3 hours and rapidly reach the necrotic core interacting with the main fungal population at 6 hours, with an average of 31.6 μg/ml drug in lesions. Sustained drug penetration and accumulation of CD101 within lesions was continuously observed for all remaining time points included in the study. Even at 72 hours following a single dose of CD101, drug levels inside lesions were still close to 30 μg/ml, about six-fold higher than that for micafungin at steady-state. The outstanding penetration of CD101 at the site of infection is dose-dependent. In the low dose (5 mg/kg) CD101 experiment, the same penetration pattern was observed, but proportionally lowered drug levels both in and outside lesions at selected time points compared to the 20 mg/kg treatment. At 24 hours after a single low dose of CD101, the mean drug concentration within lesions was 12.7 μg/ml, still about four-fold higher than what was seen with micafungin (3.4 μg/ml) at the same dosage, indicating a true superior lesion penetration feature of CD101.

The ability to quantify kinetically the level of drug at the site of infection has important implications for emergence of drug resistance. Insufficient penetration and/or drug accumulation in lesions may create temporal or spatial windows in specific niches, allowing acquisition of mutations in major drug target genes, and eventually facilitating emergence of resistance. The mutant prevention concentration (MPC) derived from the mutant selection window hypothesis, which was raised to address the need of dosing strategy to restrict emergence of resistance to antibacterial agents. MPC is the minimal concentration that inhibits drug-susceptible mutant subpopulation. The MPCs for both CD101 and micafungin are reported as 16 μg/ml against wildtype strains of C. albicans and C. glabrata. In this study, at steady-state micafungin diffused into abscesses at just under 5 μg/ml, which was above the minimum inhibitory concentration (MIC=0.03 μg/ml) but below the reported MPC. This result may help account for echinocandin treatment failures and emergence of resistance observed with some IAC patients, as drug fail below critical levels. In contrast, CD101 penetrated into the lesions as early as 6 hours after a single dose at 20 mg/kg, and drug levels were maintained exceptionally high throughout a 72-hour endpoint with a mean concentration of 29.7 μg/ml, which was well above the MPC. This suggests, under well-determined and properly designed dosing regimen, CD101 may be able to overcome or limit resistance development induced by insufficient drug penetration of currently approved echinocandin agents.

In summary, CD101 displays extraordinary penetration attributes at the site of infection relative to micafungin.

### Example 2. Treatment of IAC in a Human Subject with CD101

A human subject is diagnosed with IAC using standard diagnostic procedures. The subject is administered 400 mg to 1200 mg of the acetate salt of CD101 weekly for up to 12 weeks by intra-abdominal injection, subcutaneous injection, or intravenous infusion. The subject additionally undergoes standard source control procedures for IAC (e.g., drainage of infected material, debridement of damaged tissue, and/or surgical correction). Following the administration of CD101 and standard source control procedures, the subject is assessed in a follow-up visit and the IAC is confirmed to be resolved.

### Example 3. Treatment of IAC in a Human Subject with a Single Dose of CD101

A human subject is diagnosed with IAC using standard diagnostic procedures. The subject receives treatment with a single dose of 400 mg to 1200 mg of the acetate salt of CD101 administered by intra-abdominal injection, subcutaneous injection, or intravenous infusion. The subject undergoes standard source control procedures for IAC (e.g., surgery or drainage), but no other antifungal treatment is provided to the subject within one to three weeks before or after administration of the single dose CD101 treatment. Following the single dose administration of CD101 (e.g., one to three weeks later), the subject is assessed in a follow-up visit and the IAC is confirmed to be resolved.

### Example 4. Kidney Penetration of CD101 in C. albicans Infected Kidneys

Tissue distribution of CD101 was investigated using novel matrix-assisted laser desorption ionization mass spectrometry imaging (MALDI-MSI) in an immunocompetent invasive candidiasis mouse model.

Experimental Method

BALB/c mice were IV challenged with 2×10⁶ CFU of C. albicans strain ATCC 90028 on day 0. Single doses of CD101 at 10, 20, 40, or 60 mg/kg were administered at 24 hours post-infection via IP injection. Blood was collected pre-dose and at 1 hour, 3, 6, 12, 24, and 48 hours post-dose (3 mice/timepoint/dose) for pharmacokinetic assessment. Plasma concentrations were measured by Liquid Chromatography Mass Spectrometry/Mass Spectrometry (LC-MS/MS). Kidneys from the 10 mg/kg group at 0, 1, 3, 6, 12, and 48 h post-dose time points were analyzed for tissue distribution by MALDI-MSI.

Results

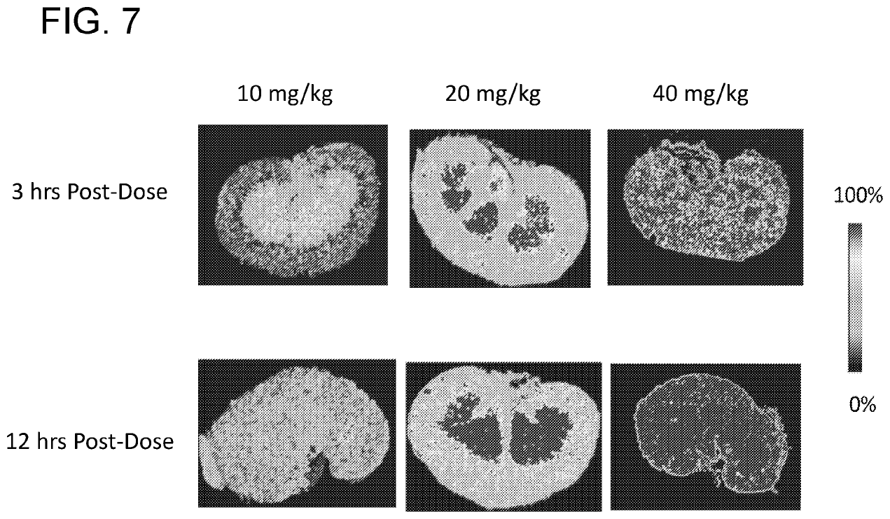

Maximum plasma concentrations (Cₘₐₓ) of CD101 were observed at 1 hour and 12 hours with mean Cₘₐₓ values of 23.1, 43.3, 82.3, and 95.8 μg/mL for doses of 10, 20, 40, and 60 mg/kg, respectively. Corresponding mean values for area under the curve (AUC)₀₋ₜ, where t=48 hours post-dose, were 736, 1250, 2380, and 3300 μg*h/mL. Mean half-life was long for each dose, ranging from 29.8 to 52.0 h. CD101 was observed with higher drug signals in the medulla but lower levels of drug reaching the outer cortex. CD101 in the kidney accumulated over time, with strong signals visualized from 3 hours to 12 hours. Drug signal decreased slowly after 12 hours post-dose, and an appreciable signal was still detectable at 48 hours at 20 mg/kg, which is the mouse equivalent human dose (Error! Reference source not found.). FIG. 7 shows a CD101 dose comparison at 3 hours and 12 hours post-dose and indicates the concentration by heat map intensity. Increase in time post-dose and increase in dose demonstrates the maximum concentration of CD101 in infected kidneys.

Conclusions

This study demonstrates that CD101 can effectively penetrate kidney tissue at the site of infection during treatment of IAC.

### Example 5. CD101 MICs and MPCs Against C. glabrata Clinical Isolates that Display a Range of Susceptibility to Anidulafungin, Caspofungin and Micafungin

Background

Echinocandins are agents of choice against most types of invasive candidiasis, but treatment failures occur in up to 40% of cases. Increased usage of the class has led to emergence of resistance, in particular among Candida glabrata. Resistance-conferring FKS gene mutations are detected in 8-18% of C. glabrata strains at certain centers. At the same time, the sizeable majority of treatment failures are not due to echinocandin resistance. The data suggest that echinocandin delivery to infected tissue sites is often insufficient to achieve concentrations that eliminate Candida or suppress resistance.

There are limited data on echinocandin pharmacokinetics-pharmacodynamics (PK-PD) at sites of Candida infection. In two studies using the mouse model of hematogenously disseminated candidiasis (DC), echinocandins persisted within kidneys and exerted ongoing anti-Candida activity after serum levels fell below minimum inhibitory concentration (MIC). These results are important, but they are not necessarily applicable to other tissues or to types of invasive candidiasis other than candidemia. Since mice were treated within hours of infection, the PK-PD results are only relevant for treatment initiated at early stages of kidney invasion, before Candida cells are sequestered within abscesses. Patients with invasive candidiasis usually are not treated this early, as diagnostic cultures require prolonged incubation and are often negative until late in disease. Indeed, scattered abscesses are the predominant histopathologic findings within kidneys and other organs from humans with invasive candidiasis. Restricted diffusion into abscesses is postulated to account, at least in part, for poor outcomes seen with delays in antifungal treatment. The most powerful echinocandin PK-PD studies would investigate abscesses or other infected lesions within tissues, rather than whole organs or serum.

Candidemia and IAC are the two most common types of invasive candidiasis, but pathogenesis and antifungal treatment of the latter is not well-studied. We have developed a mouse model of C. glabrata IAC that mimics the pathogenesis and progression of disease in humans. The model is ideally suited to study PK-PD at the site of infection because C. glabrata is localized and persists within clearly-demarcated abscesses.

Experimental Method

168 C. glabrata isolates are collected from blood or other sterile sites in our repository. 11 clinical isolates are selected that display various susceptibility profiles against anidulafungin, caspofungin, and micafungin, and/or harbor mutant or wild-type FKS genes (Table 1). Additionally, 29 isolates are picked that are susceptible to all three echinocandins and harbor wild-type FKS genes. These 40 clinical isolates undergo CD101 susceptibility testing according to the CLSI broth microdilution reference methods. Stock solutions are prepared in DMSO. The range of CD101 tested are: 0.015-16 μg/ml. MICs are determined visually after 24-h of incubation. C. krusei ATCC 6258 and C. parapsilosis ATCC 22019 are included for quality controls.

TABLE 1
Susceptibility profiles of C. glabrata isolates

| Isolate | Anidulafungin | | Caspofungin | | Micafungin | | FKS |
|---|---|---|---|---|---|---|---|
| 1 | 0.015 | (S) | 0.125 | (S) | 0.015 | (S) | R653I |
| 2 | 0.015 | (S) | 0.25 | (I) | 0.03 | (S) | Wild-type |
| 3 | 0.06 | (S) | 1 | (R) | 0.06 | (S) | F659L |
| 4 | 0.5 | (R) | 1 | (R) | 0.06 | (S) | F659S |
| 5 | 0.5 | (R) | 4 | (R) | 0.12 | (I) | R636S |
| 6 | 2 | (R) | 8 | (R) | 4 | (R) | F659del |
| 7 | 2 | (R) | 8 | (R) | 8 | (R) | S663P |
| 8 | 0.5 | (R) | 0.5 | (R) | 0.5 | (R) | S663P |
| 9 | 0.5 | (R) | 2 | (R) | 0.25 | (R) | D632Y |
| 10 | 1 | (R) | 2 | (R) | 0.25 | (R) | D632Y |
| 11 | 2 | (R) | 16 | (R) | 4 | (R) | F659del |

MPC represents a threshold above which the selective proliferation of resistant mutants is expected to occur only rarely. Thus, MPC potentially serves as a simple measure of antibiotic potency that incorporates the ability of an agent to restrict selection of resistant mutants. Five C. glabrata clinical isolates are selected that are echinocandin-susceptible and carry wild-type FKS. MPCs of CD101 and micafungin are measured using standard methods.

### Example 6. CD101 PK and Drug Distribution within Abscess and Surrounding Organ

Experimental Method

Six week-old ICR mice are infected intraperitoneally (IP) with 1×10⁸ of C. glabrata BG2 (micafungin-susceptible with MIC of 0.06 μg/mL, wild-type FKS genes) mixed with sterile stool. This clinical isolate is used to establish an IAC C. glabrata model, and it is determined that abscesses become evident after 3 days after infection. Three days after infection, mice are treated with CD101 at 60 mg/kg IV once. For drug comparison, mice are treated daily with IV micafungin for three days (5 mg/kg, a dose that achieves serum AUC similar to that of humans receiving 100-150 mg/d).

Two mice are sacrificed daily from Days 4 through 6 for CD101 PK within abscess and efficacy study (Day 3 tissue pre-treatment is used as control). The liver is focused on as the organ for tissue studies because it is large, easy to dissect and a major target for abscesses; liver tissue surrounding abscesses has low organism burdens. MALDI-MSI is used to visualize CD101 distribution and gradients within abscesses and tissue, and Grocott methenamine silver (GMS) staining to localize C. glabrata. Drug distribution and PK data are correlated with tissue burden data from previous studies, and used to derive predictions for regimens to optimize antifungal activity and limit resistance during IAC. CD101 drug concentration and distribution are also assessed in correlation with efficacy and suppression of resistance.

Other embodiments are within the claims.

What is claimed is:

1. A method of treating an individual having intra-abdominal candidiasis (IAC), comprising:
(i) identifying the individual as having IAC or suspected as having IAC; and
(ii) administering to the individual a pharmaceutical composition comprising CD101 in salt or neutral form, wherein said composition is sufficient to treat the IAC.

2. A method of preventing IAC in an individual at risk thereof, comprising:
(i) identifying the individual as being at risk of developing IAC; and
(ii) administering to the individual a pharmaceutical composition comprising CD101 in salt or neutral form, wherein said composition is sufficient to prevent the development of IAC.

3. The method of claim 1 or 2, wherein the subject has an intra-abdominal abscess (IAA) and wherein the pharmaceutical composition is administered to the individual at a dosage and frequency sufficient for the concentration of CD101 within the IAA to exceed the mutant prevention concentration (MPC) of CD101 for Candida.

4. The method of claim 3, wherein the concentration of CD101 within the IAA achieves a concentration that exceeds the MPC within 6 hours following administration.

5. The method of claims 3 or 4, wherein the concentration of CD101 within the IAA is maintained at a concentration that exceeds the MPC for at least 24 hours.

6. The method of claim 5, wherein the concentration of CD101 within the IAA is maintained at a concentration that exceeds the MPC for at least 48 hours.

7. The method of claim 6, wherein the concentration of CD101 within the IAA is maintained at a concentration that exceeds the MPC for at least 72 hours.

8. The method of any one of claims 3-7, wherein the MPC is 1 μg/ml to 30 μg/ml.

9. The method of claim 8, wherein the MPC is about 16 μg/ml.

10. The method of any one of claims 1-9, wherein the pharmaceutical composition is administered subcutaneously, intra-abdominally, or intravenously.

11. The method of any one of claims 1-10, wherein the pharmaceutical composition is administered weekly for one to twelve weeks in an amount of 200 to 1200 mg.

12. The method of any one of claims 1-11, wherein the pharmaceutical composition is administered is administered to the subject in a single dose, wherein the single dose comprises an amount of the CD101 in salt or neutral form sufficient to treat IAC.
